(19) United States
(12) Patent Application Publication
(10) Pub. No.: US 2025/0160319 A1
(43) Pub. Date: May 22, 2025

(54) STABILIZATION OF THROMBOCYTES AT AMBIENT TEMPERATURES

(71) Applicant: Biomatrica, Inc., San Diego, CA (US)

(72) Inventors: Rolf MULLER, Del Mar, CA (US); Joel DESHARNAIS, La Mesa, CA (US); Steven P. WILKINSON, San Diego, CA (US); Victoria ARENDT, San Diego, CA (US); Paul DIAZ, Riverside, CA (US)

(21) Appl. No.: 18/890,348

(22) Filed: Sep. 19, 2024

Related U.S. Application Data
(63) Continuation of application No. 18/202,419, filed on May 26, 2023, now Pat. No. 12,121,022.
Application No. 18/202,419 is a continuation of application No. 16/931,698, filed on Jul. 17, 2020, now Pat. No. 11,672,247.
Application No. 16/931,698 is a continuation of application No. 16/049,516, filed on Jul. 30, 2018, now Pat. No. 10,772,319.
Application No. 16/049,516 is a continuation of application No. 15/316,677, filed on Dec. 6, 2016, now Pat. No. 10,064,404.
(60) Provisional application No. 62/010,151, filed on Jun. 10, 2014.

(51) Int. Cl. A01N 1/126; A01N 1/122; A61K 35/19

(52) U.S. Cl. CPC A01N 1/126; A01N 1/122; A61K 35/19

(57) ABSTRACT

Provided herein are formulations and methods for the stabilization of one or more thrombocytes at ambient temperatures. Also provided are formulations and methods for the stabilization of one or more thrombocytes in an inactivated state in a blood sample at ambient temperatures. Further provided are articles of manufacture and kits and methods for substantially stable storage of one or more thrombocyte at ambient temperatures.

### CROSS-REFERENCE

This application is a continuation of U.S. patent application Ser. No. 18/202,419, filed May 26, 2023, which is a continuation of U.S. patent application Ser. No. 16/931,698, filed Jul. 17, 2020, now U.S. Pat. No. 11,672,247, which is a continuation of U.S. patent application Ser. No. 16/049,516, filed Jul. 30, 2018, now U.S. Pat. No. 10,772,319, which is a continuation of U.S. patent application Ser. No. 15/316,677, filed Dec. 6, 2016, now U.S. Pat. No. 10,064,404, which is a U.S. National Phase Application of International Patent Application No. PCT/US2015/034967, filed Jun. 9, 2015, which claims the benefit of U.S. Provisional Application No. 62/010,151, filed Jun. 10, 2014, all of which are incorporated by reference herein in their entirety.

### BACKGROUND OF THE INVENTION

### 1. Technical Field

The present invention relates generally to stabilization of one or more thrombocytes at ambient temperatures. In particular, the invention relates to formulations, compositions, articles of manufacture, kits and methods for substantially stable storage of one or more metabolically- active thrombocytes at ambient temperatures.

### Background

Whole blood is a complex mixture of cells, nucleic acids, proteins and various other analytes. In particular, blood components include, but are not limited to: cells, such as leukocytes (monocytes, lymphocytes and granulocytes), erythrocytes, thrombocytes and circulating tumor cells; nucleic acid molecules, such a circulating-free DNA (cfDNA); polypeptides, such as lipoproteins, albumin and serum proteins, and other various analytes.

Thrombocytes or platelets are anucleated cells that play a key role in the clotting of blood. Thrombocytes are small, disc-like cells that circulate in mammalian blood and are involved in hemostasis. Thrombocytes secrete a wide variety of growth factors that assist in promoting blood clotting and tissue regeneration.

The level of circulating thrombocytes in a healthy individual is controlled within a physiological range of about (150-400)×10³ per mm³. Suboptimal levels of thrombocytes (thrombocytopenia) can lead to excessive bleeding, whereas levels exceeding optimal concentrations can lead to the formation of thromboli (blood clots) that can obstruct blood vessels and can lead to higher risk of stroke, pulmonary embolus or myocardial infarction.

Circulating thrombocytes are typically present in an inactivated state, and are maintained in the inactivated state by factors produced by endothelial cells lining the blood vessel lumen. Upon disruption or injury to this endothelial layer, thrombocytes come in contact with collagen or von Wildebrand's factor, which activates the thrombocytes causing the thrombocytes to aggregate (i.e., clot). This activation and aggregation also may occur by the enzymatic activity of thrombin or in the presence of ADP. Upon activation, thrombocytes release the contents of alpha and dense granules that include growth factors and fibrinogen that assist in clot formation and help promote recruitment of fibroblasts to promote wound healing. Activated thrombocytes can be distinguished from inactivated thrombocytes by their more spherical/stellate shape.

The activation, aggregation and/or release of numerous growth factors and other intracellular components of thrombocytes during the collection of whole blood can greatly hinder the quantitation and analysis of these cells. The addition of various anti-coagulants to maintain an inactivated thrombocytes at ambient temperatures results in only about 13-52% inactivated thrombocytes at 24 hours making accurate quantitative analysis of total thrombocytes essentially impossible at this time point. Thus, there exists a need for improved formulations for and methods of stabilizing thrombocytes at ambient temperatures for a time sufficient for storage and shipping thrombocytes for research, diagnostic and therapeutic purposes.

### SUMMARY OF THE INVENTION

The formulations, compositions and methods of the present invention advantageously provide for the stabilization of thrombocytes at ambient temperatures and these cells remain functional and retain the ability to be activated post-blood collection for a period of at least 24 hours, significantly increasing the time for storage and shipping of substantially stable thrombocytes for research, diagnostic and potential therapeutic applications. Disclosed herein in some embodiments, are formulations for substantially stable storage of one or more thrombocytes at ambient temperatures, wherein the one or more thrombocytes are stabilized for a period of at least six hours. In some embodiments, the one or more thrombocytes are stabilized in an inactivated state. In some embodiments, the one or more thrombocytes are in a blood sample. In some embodiments, the one or more thrombocytes are in an inactivated state in a blood sample. In some embodiments, the one or more thrombocytes are isolated from a blood sample. In some embodiments, at least 90% of the thrombocytes are stabilized in an inactivated state for a period of at least six hours. In some embodiments, at least 90% of the thrombocytes are stabilized in an inactivated state for a period of at least nine hours. In some embodiments, the thrombocytes are stabilized in an inactivated state for a period of at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours. In some embodiments, the formulation comprises: (i) a pH buffer; (ii) an anti-coagulant; (iii) at least one non-reducing sugar or polyol; and (iv) a functionalized carbohydrate. In some embodiments, the formulation comprises a polyol selected from the group consisting of glycol, glycerol, erythritol, threitol, arabitol, xylitol, ribitol, adonitol, mannitol, sorbitol, galactitol, fucitol, iditol and inositol, and combinations thereof. In some embodiments, the polyol is a pentose polyol or a hexose polyol. In some embodiments, the pentose polyol is adonitol. In some embodiments, the functionalized carbohydrate is sucralfate or sucrose octasulfate. In some embodiments, the functionalized carbohydrate is sucrose octasulfate. In some embodiments, the non-reducing sugar is sucrose or trehalose. In some embodiments, the non-reducing sugar is trehalose. In some embodiments, the anticoagulant is EDTA or hirudin. In some embodiments, the pH buffer is 2×phosphate buffered saline or Tris-HCl.

In one aspect of the invention, formulations are provided for substantially stable storage of one or more thrombocytes in an inactivated state in a blood sample at ambient temperatures, wherein the one or more thrombocytes are stabilized in an inactivated state for a period of at least six hours. In some embodiments, at least 90% of the thrombocytes are stabilized in an inactivated state for a period of at least six hours. In some embodiments, the thrombocytes are stabilized in an inactivated state for a period of at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours. In certain embodiments, the formulation comprises (i) a pH buffer, (ii) an anticoagulant, (iii) at least one non-reducing sugar or polyol, and (iv) a functionalized carbohydrate. In some embodiments, the polyol is selected from the group consisting of glycol, glycerol, erythritol, threitol, arabitol, xylitol, ribitol, adonitol, mannitol, sorbitol, galactitol, fucitol, iditol inositol, and combinations thereof. In some embodiments the polyol is a pentose polyol or a hexose polyol. In some embodiments, the polyol is adonitol. In some embodiments, the functionalized carbohydrate is sucralfate or sucrose octasulfate. In some embodiments, the functionalized carbohydrate is sucrose octasulfate. In some embodiments, the non-reducing sugar is sucrose or trehalose. In some embodiments, the non-reducing sugar is trehalose. In some embodiments, the anticoagulant is EDTA or hirudin. In some embodiments, the anticoagulant is EDTA, the functionalized carbohydrate is sucralfate or sucrose octasulfate, and the non-reducing sugar is sucrose or trehalose. In yet other embodiments, the anticoagulant is EDTA, the functionalized carbohydrate is sucrose octasulfate and the non-reducing sugar is trehalose. In some embodiments, the pH buffer is 2×phosphate buffered saline or Tris-HCl. Disclosed herein, in some embodiments are formulations for substantially stable storage of one or more thrombocytes at ambient temperatures, comprising a halogenated disaccharide derivative and an anticoagulant, wherein the one or more thrombocytes are stabilized for a period of at least six hours. In some embodiments, the one or more thrombocytes are stabilized in an inactivated state. In some embodiments, the one or more thrombocytes are in a blood sample. In some embodiments, the one or more thrombocytes are stabilized in an inactivated state in a blood sample. In some embodiments, the one or more thrombocytes are isolated from a blood sample. In some embodiments, the anticoagulant is hirudin. In some embodiments, the halogenated disaccharide derivative is selected from the group consisting of sucralose (1,6-dichloro-1,6-dideoxy-β-D-fructofuranosyl-4-chloro-4-deoxy-α-D-galactopyranoside), trichloronated maltose, 1,6-dichloro-1,6-dideoxy-β-D-fructofuranosyl-4-chloro-4-deoxy-6-O-monododecanoate-α-D-galactopyranoside, 1,6-sichloro-1,6-dideoxy-β-D-fructofuranosyl-4-chloro-4-deoxy-6-O-monotetradecanoate-α-D-galactopyranoside, and combinations thereof. In some embodiments, the formulation consists essentially of hirudin and sucralose (1,6-dichloro-1,6-dideoxy-β-D-fructofuranosyl-4-chloro-4-deoxy-α-D-galactopyranoside).

In some embodiments, there are provided formulations for substantially stable storage of one or more thrombocytes in an inactivated state in a blood sample at ambient temperatures, comprising a halogenated disaccharide derivative, wherein the one or more thrombocytes are stabilized in an inactivated state for a period of at least six hours. In some embodiments, the halogenated disaccharide derivative preferably is selected from the group consisting of sucralose (1,6-dichloro-1,6-dideoxy-β-D-fructofuranosyl-4-chloro-4-deoxy-α-D-galactopyranoside), trichloronated maltose, 1,6-dichloro-1,6-dideoxy-β-D-fructofuranosyl-4-chloro-4-deoxy-6-O-monododecanoate-α-D-galactopyranoside and 1,6-dichloro-1,6-dideoxy-β-D-fructofuranosyl-4-chloro-4-deoxy-6-O-monotetradecanoate-α-D-galactopyranoside, and more preferably the halogenated disaccharide derivative is sucralose (1,6-dichloro-1,6-dideoxy-β-D-fructofuranosyl-4-chloro-4-deoxy-α-D-galactopyranoside). In some embodiments, the formulations further comprise an anticoagulant, preferably hirudin. In some embodiments, the anticoagulant is hirudin. In some embodiments, the formulation consists essentially of hirudin and sucralose (1,6-dichloro-1,6-dideoxy-β-D-fructofuranosyl-4-chloro-4-deoxy-α-D-galactopyranoside).

Disclosed herein in some embodiments, are compositions of substantially, stably stored one or more thrombocytes comprising one or more thrombocytes admixed with a disclosed formulation. In some embodiments, the one or more thrombocytes are in a blood sample. In some embodiments, the one or more thrombocytes are isolated thrombocytes. In some embodiments, the one or thrombocytes are in an inactivated state.

Disclosed herein in some embodiments, are articles of manufacture, comprising a formulation described herein contained within a blood collection tube. In some embodiments, the blood collection tube is an evacuated blood collection tube.

Disclosed herein, in some embodiments, are kits comprising an article of manufacture described herein and a package insert.

Disclosed herein in some embodiments, are methods for substantially stable storage of one or more thrombocytes at ambient temperatures, comprising: admixing the one or more thrombocytes from a subject with a formulation provided herein, wherein the one or more thrombocyte is stabilized for a period of at least six hours. In some embodiments, the one or more thrombocytes are stabilized in an inactivated state. In some embodiments, the one or more thrombocytes are in a blood sample from the subject. In some embodiments, the one or more thrombocytes are stabilized in an inactivated state in a blood sample from the subject. In some embodiments, the one or more thrombocytes are isolated from a blood sample from the subject. In some embodiments, at least 90% of the thrombocytes are stabilized in an inactivated state for a period of at least six hours. In some embodiments, at least 90% of the thrombocytes are stabilized in an inactivated state for a period of at least nine hours. In some embodiments, the method further comprises activating the one or more thrombocyte in an inactivated state, by the addition of an activating agent to promote thrombocyte aggregation. In some embodiments, the activating agent is ADP. In some embodiments, the method further comprises activating the one or more thrombocyte by the addition of an activating agent to promote thrombocyte aggregation. In some embodiments, the activating agent is ADP. In some embodiments, the subject is an animal. In some embodiments, the subject is a mammal. In some embodiments, the mammal is a human.

Disclosed herein in some embodiments, are methods for substantially stable storage of one or more thrombocyte in an inactivated state in a blood sample at ambient temperatures, comprising, admixing a blood sample from a subject with a formulation provided herein, wherein the one or more thrombocytes are stabilized in an inactivated state for a period of at least six hours. In some embodiments, at least 90% of the thrombocytes are stabilized in an inactivated state for a period of at least six hours. In some embodiments, the thrombocytes are stabilized in an inactivated state for a period of at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours. In some embodiments, the blood sample is admixed with the stabilization formulation at the time the blood sample is collected from the subject to substantially stabilize the one or more thrombocytes in the inactived state post collection from the subject. In some embodiments, the method further comprises activating the one or more thrombocytes by the additional of an activating agent. In some embodiments, the activating agent is ADP. In further embodiments of the methods, the subject is an animal, more preferably a mammal, and even more preferably a human.

### DETAILED DESCRIPTION OF THE INVENTION

The present invention relates to formulations, compositions, articles of manufacture, kits, and methods for substantially stable storage of one or more thrombocyte at ambient temperatures. In some embodiments, the one or more thrombocytes are stored in an inactivated, but activatable, state in a blood sample. In one aspect, the formulations described herein beneficially maintain the integrity of inactivated, metabolically active, thrombocytes that may be subsequently analyzed for activation or that may be used in therapeutic applications for promoting blood clotting in a patient.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

Unless defined otherwise, all technical and scientific terms used herein have the same meaning as is commonly understood by one of skill in the art to which this invention belongs. All patents, patent applications and publications referred to herein are incorporated by reference in their entirety.

Formulations are provided, in some embodiments, for substantially stable storage of metabolically-active thrombocytes at ambient temperatures. In some embodiments, the thrombocytes are isolated from a blood sample. In some embodiments, the thrombocytes are in a blood sample. In some embodiments, the thrombocytes are inactivated. In certain embodiments, the thrombocyte stabilization formulations comprise a pH buffer, an anticoagulant, a non-reducing sugar, a polyol, and a functionalized carbohydrate. In certain other embodiments, the thrombocyte stabilization formulations comprise a pH buffer, an anticoagulant, a polyol and a functionalized carbohydrate. In certain other embodiments, the stabilization formulations comprise a pH buffer, an anticoagulant, a non-reducing sugar, and a functionalized carbohydrate. In still yet another embodiment, the stabilization formulations comprise a halogenated disaccharide derivative and an anticoagulant. In still yet another embodiment, the stabilization formulations comprise a halogenated disaccharide derivative, an anticoagulant, and a pH buffer. The formulations are capable of stabilizing at least 60%, 70%, 80% or even 90% inactivated, metabolically-active thrombocytes in a blood sample at ambient temperatures for a period of at least 6 hours, or at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours.

The term “ambient temperature” as used herein refers to common indoor room temperatures. In some embodiments, ambient temperature is 15 to 32° C. In some embodiments, ambient temperature is 20 to 27° C.

In another aspect of the present invention, formulations are provided for substantially stable storage of inactivated, metabolically-active thrombocytes in a blood sample at ambient temperatures. In certain embodiments, the thrombocyte stabilization formulations comprise a pH buffer, an anticoagulant, a non-reducing sugar, a polyol, and a functionalized carbohydrate. In certain other embodiments, the thrombocyte stabilization formulations comprise a pH buffer, an anticoagulant, a polyol, and a functionalized carbohydrate. In certain other embodiments, the stabilization formulations comprise a pH buffer, an anticoagulant, a non-reducing sugar, and a functionalized carbohydrate. In still yet another embodiment, the stabilization formulations comprise a halogenated disaccharide derivative and an anticoagulant. In still yet another embodiment, the stabilization formulations comprise a halogenated disaccharide derivative, an anticoagulant, and a pH buffer. The formulations are capable of stabilizing at least 60%, 70%, 80% or even 90% inactivated, metabolically-active thrombocytes in a blood sample at ambient temperatures for a period of at least 6 hours, or at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours.

In another aspect, compositions are provided herein in which a blood sample is admixed with a thrombocyte stabilization formulation to produce substantially stable one or more inactivated thrombocytes in a whole blood preparation. In still other embodiments, a composition comprising purified or substantially purified one or more thrombocyte admixed with a stabilization formulation of the present invention are provided.

### Formulation Reagents

### A. pH Buffers

According to certain embodiments, the herein described formulations and compositions for substantially stable storage of one or more thrombocytes include one or more pH buffers. In some embodiments, the pH buffer is any of a large number of compounds known in the art for their ability to resist changes in the pH of a solution, such as in an aqueous solution in which the pH buffer is present. Selection of one or more particular pH buffers for inclusion in a stable storage composition may be done based on the present disclosure and according to routine practices in the art, and may be influenced by a variety of factors including the pH that is desired to be maintained, the nature of the biological sample, the solvent conditions to be employed, the other components of the formulation to be used, and other criteria. For example, typically a pH buffer is employed at a pH that is within about 0.1, 0.2, 0.3, 0.4, 0.5, 0.6, 0.7, 0.8, 0.9 or 1.0 pH unit of a proton dissociation constant (pKₐ) that is a characteristic of the buffer.

Non-limiting examples of pH buffers include citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane) and bis-tris (2-[bis(2-hydroxyethyl)amino]-2-(hydroxymethyl)-1,3-propanediol). In some embodiments, including any of those set forth in Table 1, have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

### B. Polyols

Also as described herein, certain embodiments include at least one polyol in the composition for substantially stable storage of viable, inactivated thrombocytes in a whole blood sample at ambient temperatures. Polyols are polyhydric alcohols containing two or more hydroxyl groups and have the general formula H(CHOH)ₙH, wherein n is an integer selected from 2 to 7 inclusive. Polyols differ in chain length with most polyols having five-or six carbon chains being derived from pentoses (five-carbon sugars) and hexoses (six-carbon sugars); however shorter and longer carbon chain polyols also exist. Exemplary polyols include, but are not limited to, glycol, glycerol, erythritol, threitol, arabitol, xylitol, ribitol, adonitol, mannitol, sorbitol, galactitol, fucitol, iditol and inositol. Selection of one or more particular polyols for inclusion in a substantially stable storage composition may be done based on the present disclosure and according to routine practices in the art, and may be influenced by a variety of factors including other formulation components. In certain embodiments, the polyol present in the formulation is a pentose polyol. In some embodiments, the polyol is adonitol. In some embodiments, the polyol is present at a concentration between 20-100 mM, or between about 25-75 mM. In some embodiments, the polyol is a pentose polyol and is present at a concentration between 20-100 mM, or between about 25-75 mM. In some embodiments, the polyol is adonitol and is present at a concentration between 20-100 mM, or between about 25-75 mM.

### C. Disaccharide Derivatives

In certain embodiments, the formulations or compositions for substantially stable storage of one or more inactivated thrombocyte in a whole blood sample at ambient temperatures, including those in Table 1, include at least one halogenated disaccharide derivative. In some embodiments, the halogenated disaccharide derivative is a di- or tri-chlorinated disaccharide. In some embodiments, such di- or tri-chlorinated disaccharides unexpectedly are capable of substantially stable storage of inactivated thrombocytes either alone or in the presence of only a buffer. Halogenated disaccharide derivatives are known, e.g., see U.S. Patent Publication No. 2014/0065062, and include sucralose (1,6-dichloro-1,6-dideoxy-β-D-fructofuranosyl-4-chloro-4-deoxy-α-D-galactopyranoside), trichloronated maltose, 1,6-dichloro-1,6-dideoxy-β-D-fructofuranosyl-4-chloro-4-deoxy-6-O-monododecanoate-α-D-galactopyranoside, and 1,6-dichloro-1,6-dideoxy-β-D-fructofuranosyl-4-chloro-4-deoxy-6-O-monotetradecanoate-α-D-galactopyranoside. Selection of one or more particular halogenated disaccharide derivative for inclusion in a substantially stable storage composition may be done based on the present disclosure and according to routine practices in the art, and may be influenced by a variety of factors including other formulation components. In some embodiments, the functionalized carbohydrate is sucralose and is present at about 1.0-50.0 mM. In some embodiments, the functionalized carbohydrate is sucralose and is present at about 10.0-30.0 mM. In some embodiments, the functionalized carbohydrate is sucralose and is present at about 25.0 mM.

### D. Functionalized Carbohydrates

In some embodiments described herein, the formulations, including those in Table 1, include a functionalized carbohydrate. Exemplary functionalized carbohydrates include sucralfate or sucrose octasulfate and it will be appreciated that from the present disclosure the skilled person may select other functionalized carbohydrates for use in a stable storage formulations and compositions for viable, activatable, thrombocytes, as may vary based on the other components of the composition that are employed. In some embodiments, the concentration of functionalized carbohydrates in the present formulations and compositions, including those set forth in Table 1, is about 0.005-1.0 mM. In some embodiments, the concentration of functionalized carbohydrates in the present formulations and compositions, including those set forth in Table 1, is about 0.25-0.5 mM.

### E. Non-Reducing Sugars

In some embodiments, the formulations and compositions for substantially stable storage of thrombocytes at ambient temperatures include at least one non-reducing sugar. In some embodiments, the formulations and compositions for substantially stable storage of viable, inactivated thrombocytes in a whole blood sample at ambient temperatures include at least one non-reducing sugar. As used herein, “non-reducing sugars” refers to carbohydrate molecules that lack a functional aldehyde group. Exemplary non-reducing sugars include sucrose and trehalose. In some embodiments, the non-reducing sugar is sucrose. In some embodiments, the non-reducing sugar is trehalose. In some embodiments, the trehalose is present at a concentration of about 1.0-50 mM. In some embodiments, the trehalose is present at a concentration of about 10.0-30 mM. In some embodiments, the trehalose is present at a concentration of about 25 mM.

### F. Anticoagulants

In some embodiments, an anticoagulant is included in the presently described formulations or compositions. Such anticoagulants are known in the art. Exemplary anticoagulants include ethylenediaminetetraacetic acid (EDTA), hirudin, heparin, and sodium citrate. In some embodiments, the anticoagulant is hirudin. In some embodiments, the hirudin is present at a concentration of about 1.0-50 μg/mL. In some embodiments, the hirudin is present at a concentration of about 1.0-25 μg/mL. In some embodiments, the hirudin is present at a concentration of about 10-20 μg/mL.

### Exemplary Formulations for Stabilization of Thrombocytes at Ambient Temperatures

In some embodiments, the formulations, compositions and methods of the present invention advantageously provide for the substantially stable storage of thrombocytes at ambient temperatures for a period of at least six hours. In some embodiments, the formulations, compositions and methods of the present invention advantageously provide for the substantially stable storage of thrombocytes in their natural circulating, inactivated state in a blood sample at ambient temperatures, wherein the cells retain the ability to be activated post collection for a period of at least six hours. In other embodiments, at least 90% of the thrombocytes are stabilized in an inactivated state for a period of at least six hours. In other embodiments, the thrombocytes are stabilized in an inactivated state for a period of at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours.

In certain embodiments, the formulations for the substantially stable storage of thrombocytes at ambient temperatures comprise a pH buffer, an anticoagulant, a non-reducing sugar, a polyol, and a functionalized carbohydrate. In certain embodiments, the stabilization formulations comprise a pH buffer, an anticoagulant, a polyol, and a functionalized carbohydrate. In certain embodiments, the formulations for the substantially stable storage of thrombocytes comprise a pH buffer, an anticoagulant, a non-reducing sugar, and a functionalized carbohydrate. In some embodiments, the formulations for the substantially stable storage of thrombocytes comprise a halogenated disaccharide derivative, and an anticoagulant, and may further comprise a pH buffer. In some embodiments, the anti-coagulant is sprayed and dried on the blood collection tube, container, or vessel prior to collection of the blood sample from the subject. In some embodiments, the anti-coagulant is added directly to the formulations described herein.

In certain embodiments, the pH buffer is Tris-HCl, the polyol is a pentose alcohol, the non-reducing sugar is trehalose, the anticoagulant is EDTA or hirudin, and the functionalized carbohydrate is sucrose octasulfate. In certain embodiments, the pH buffer is Tris-HCl, the polyol is adonitol, the non-reducing sugar is the D+ isomer of trehalose, the anticoagulant is EDTA or hirudin, and the functionalized carbohydrate is sucrose octasulfate. In certain embodiments, the pH buffer is Tris-HCl, the polyol is adonitol, the non-reducing sugar is the D+ isomer of trehalose, the anticoagulant is hirudin, and the functionalized carbohydrate is sucrose octasulfate. In some embodiments, the disaccharide derivative is a halogenated disaccharide and the anticoagulant is hirudin. In some embodiments, the halogenated disaccharide is sucralose and the anticoagulant is hirudin.

In some embodiments, the formulations for the substantially stable storage of inactivated thrombocytes at ambient temperatures include the exemplary formulations provided in Table 1.

TABLE 1
Exemplary Formulations for Stabilizing Inactivated,
Metabolically-active Thrombocytes in a Human
Blood Sample at Ambient Temperatures

| | Tris- | | | Sucrose | |
|---|---|---|---|---|---|
| | HCl | Adonitol | Trehalose | Octasulfate | Sucralose |
| Formulation | (mM) | (mM) | (mM) | (mM) | (mM) |
| A | 2.5 | 100 | | 1.0 | |
| B | 2.5 | 50 | 25.0 | 1.0 | |
| C | 2.5 | | 25.0 | 1.0 | |
| D | 2.5 | 50 | 25.0 | 0.5 | |
| E | 2.5 | | | | 25 |
| F | | | | | 25 |

### Methods for Preparing Exemplary Formulations

In some embodiments, the exemplary Formulations A-F of Table 1 are prepared using materials commercially available from suppliers and preparing such formulations is accomplished using the methods disclosed herein as well as other methods known to those skilled in the art.

In some embodiments, pre-weighed solid components are added to a suitable vessel, such as a square bottle, to which the aqueous components are added. The reaction mixture is agitated, e.g., by shaking, until the solid components have completely dissolved and then the pH of the mixture is adjusted to the desired pH using a suitable acid, e.g., hydrochloric acid. The resulting formulations are then sterilized, e.g., using a 0.22 micron filter, and stored at room temperature.

In one example, a 50 mL preparation of a 20×Formula A is prepared as follows: 15.2034 gr of adonitol (Calbiochem, catalogue #121739) and 1.251 g sucrose octasulfate potassium salt (Toronto Research Chemicals, catal, ogue #S69900) are added to a square bottle. A 30 mL volume of water is added, followed by the addition of 2.5 mL of Tris-HCl (Invitrogen, catalogue #15567-027). Additional water is added to qc the formulation to a final total volume of 50 mL. The mixture is shaken until fully dissolved, and hydrochloric acid is added to adjust the pH to 7.51. The solution is sterile filtered (0.22 μm pore size) under vacuum to yield the resulting formulation.

In another example, a 50 mL preparation of a 20×Formula B is prepared as follows: 7.5994 g of adonitol, 9.4997 g D-(+)-trehalose dihydrate (Fluka, catalogue #90210), and 1.25 g sucrose octasulfate potassium salt are added to a square bottle. A 30 mL volume of water is added, followed by the addition of 2.5 mL of Tris-HCl (Invitrogen, catalogue #15567-027). Additional water is added to qc the formulation to a final total volume of 50 mL. The mixture is shaken until fully dissolved, and hydrochloric acid is added to adjust the pH to 7.51. The solution is sterile filtered (0.22 μm pore size) under vacuum to yield the resulting formulation.

In another example, a 50 mL preparation of a 20×Formula C is prepared as follows: 9.5003 g D-(+)-trehalose dehydrate and 1.2502 g sucrose octasulfate potassium salt are added to a square bottle. A 30 mL volume of water is added, followed by the addition of 2.5 mL of Tris-HCl (Invitrogen, catalogue #15567-027). Additional water is added to qc the formulation to a final total volume of 50 mL. The mixture is shaken until fully dissolved, and hydrochloric acid is added to adjust the pH to 7.52. The solution is sterile filtered (0.22 μm pore size) under vacuum to yield the resulting formulation.

In another example, a 50 mL preparation of a 20×Formula D is prepared as follows: 7.5994 g of adonitol, 9.5003 g D-(+)-trehalose dihydrate and 0.625 g sucrose octasulfate potassium salt. A 30 mL volume of water is added, followed by the addition of 2.5 mL of Tris-HCl (Invitrogen, catalogue #15567-027). Additional water is added to qc the formulation to a final total volume of 50 mL. The mixture is shaken until fully dissolved, and hydrochloric acid is added to adjust the pH to 7.51. The solution is sterile filtered (0.22 μm pore size) under vacuum to yield the resulting formulation.

In another example, a 50 mL preparation of a 20×Formula E is prepared as follows: 9.9997 g of sucralose (Sigma, catalogue #69293) is added to a square bottle. A 30 mL volume of water is added, followed by the addition of 2.5 mL of Tris-HCl (Invitrogen, catalogue #15567-027). Additional water is added to qc the formulation to a final total volume of 50 mL. The mixture is shaken until fully dissolved and hydrochloric acid is added to adjust the pH to 7.54. The solution is sterile filtered (0.22 μm pore size) under vacuum to yield the resulting formulation.

In another example, a 50 mL preparation of a 20×Formula F is prepared as follows: 9.9993 g of sucralose (Sigma, catalogue #69293) is added to a square bottle. Water is added to qc the formulation to a final total volume of 50 mL. The mixture is shaken until fully dissolved, providing a solution having a pH of 7.54. The solution is sterile filtered (0.22 μm pore size) under vacuum to yield the resulting formulation.

### Purified Stabilized Thrombocytes

In some embodiments, the substantially stabilized one or more thrombocytes in a blood sample at ambient temperatures are purified using well known methods employed by those skilled in the art. Apparatus and kits for purifying thrombocytes from blood are well known (e.g., see U.S. Pat. Nos. 5,234,593; 6,315,706 and 7,708,152). In certain embodiments, the thrombocytes are purified using a bag PC (platelet concentrate) prepared after collection of blood in a bag and the apheresis PC obtained by the use of a component blood collecting device. These methods separate thrombocytes from blood using centrifugal separation. In some embodiments, substantially stabilized, intact, metabolically active viable cells are advantageously purified by affinity chromatography or by fluorescence activated cell sorting (FACS) analysis using antibodies generated against a native wild type membrane proteins and receptors, and which method is not possible using other storage formulations that denature these cellular proteins.

In some embodiments, the purified one or more thrombocyte are subsequently stored in the formulations described herein for extended periods before analysis or use.

### Articles of Manufacture

In certain embodiments, articles of manufacture are provided, which comprise a formulation provided herein, contained within a suitable blood collection tube, container or vessel. In some embodiments, the formulation is selected from those set forth in Table 1. In some embodiments, these articles of manufacture are used for substantially stable storage of one or more blood component by stabilizing one or more blood component at the time of blood collection. In certain embodiments, the blood collection tube is an evacuated blood tube having less than atmospheric pressure to withdraw a predetermined volume of whole blood. In some embodiments, these articles of manufacture are used in the kits and methods described herein.

### Kits

In certain embodiments, there are provided kits comprising any one of the articles of manufacture described herein and a package insert. In some embodiments, the components of the kit are supplied in a packaging means, such as a compartmentalized plastic enclosure, preferably with a hermetically sealable cover so that the contents of the kit can be sterilized and sealed for storage.

### Methods for Substantially Stable Storage of One or More Thrombocyte in a Blood Sample at Ambient Temperatures

Described herein, in some embodiments, are methods for substantially stable storage of one or more thrombocyte at ambient temperatures. In some embodiments, the methods are for substantially stable storage of one or more thrombocytes in an inactivated state in a blood sample at ambient temperatures.

In certain embodiments, the methods comprise admixing a blood sample with a formulation for substantially stable storage of one or more thrombocyte at ambient temperatures for a period of at least six hours. In some embodiments, the one or more thrombocytes are isolated from a blood sample. In some embodiments, the one or more thrombocytes are stabilized in an inactivated state. In some embodiments, at least 90% of the thrombocytes remain in an inactivated state for a period of at least six hours. In other embodiments, the thrombocytes are stabilized in an inactivated state for a period of at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours. In certain embodiments, the formulation is one of the formulations set forth in Table 1.

In certain embodiments, the methods comprise admixing a blood sample with a formulation for substantially stable storage of viable thrombocytes, wherein the formulation comprises a pH buffer, an anticoagulant, a non-reducing sugar, a polyol and a functionalized carbohydrate. In certain embodiments, the thrombocyte stabilization formulations comprise a pH buffer, an anticoagulant, a polyol, and a functionalized carbohydrate. In certain embodiments, the stabilization formulation comprises a pH buffer, an anticoagulant, a non-reducing sugar, and a functionalized carbohydrate. In still yet another embodiment, the stabilization formulation comprises a halogenated disaccharide derivative and an anticoagulant. In still yet another embodiment, the stabilization formulation comprises a halogenated disaccharide derivative and an anticoagulant furthers comprise a pH buffer. In certain embodiments, the formulation is one of the formulations set forth in Table 1.

In certain embodiments, the methods comprise admixing a blood sample with a formulation for substantially stable storage of viable, activatable thrombocytes in a blood sample, wherein the formulation comprises a pH buffer, an anticoagulant, a non-reducing sugar, a polyol, and a functionalized carbohydrate. In certain embodiments, the thrombocyte stabilization formulation comprises a pH buffer, an anticoagulant, polyol, and a functionalized carbohydrate. In certain other embodiments, the stabilization formulation comprises a pH buffer, an anticoagulant, a non-reducing sugar, and a functionalized carbohydrate. In still yet another embodiment, the stabilization formulation comprises a halogenated disaccharide derivative and an anticoagulant. In still yet another embodiment, the stabilization formulation further comprises a pH buffer. In certain embodiments, the formulation is one of the formulations set forth in Table 1.

Blood collection tubes, bags, containers and vessels are well-known in the art and have been employed by medical practitioners for decades. Blood collected for substantially stable storage of one or more blood component may be obtained from a subject, donor, or patient using any method or apparatus commonly employed by those skilled in the art such as venipuncture or finger prick. In some embodiments, when the blood is collected by venipuncture, a formulation described herein is located inside the blood collection tube, e.g., an evacuated tube (Vacutainer, Becton Dickenson or Vacuette, Greiner) at the time that the blood sample is obtained from the donor or patient. In some embodiments, the stabilization formulation is added to an already obtained whole blood sample, preferably immediately or shortly after it is withdrawn.

In some embodiments, the methods described herein use the articles of manufacture and kits disclosed.

The following Examples are presented by way of illustration and not limitation.

### Example 1: Stabilization of Inactive Thrombocytes in a Human Blood Sample for a Period of at Least 22 Hours at Ambient Temperature

This Example describes formulations of the present invention for stabilizing inactivated thrombocytes that remain capable of being activated after being stored for a period of 22 hours at ambient temperatures.

Whole blood samples were collected from six human donors using commercially available hirudin-coated collection tubes (Roche Diagnostics), the blood samples were pooled and within three hours of collection, blood samples were processed. A 300 μL aliquot of each whole blood sample was transferred to an Eppendorf tube at a 1:20 ratio with 15 μL of stabilizer formulation A, B, C, or D of Table 1, either prior to or following the addition of the stabilizer formulation, and the mixtures were kept at ambient temperatures for predetermined time periods before being analyzed. An equal volume of whole blood was added to each control sample, and each sample was stored at room temperature in the absence of the stabilizer formulation and processed in parallel with the test samples.

To 300 μL of each mixture and control, 300 μL of NaCl 0.9% and 20 μL of the provided ADP solution was added to promote activation of the thrombocytes and the samples were analyzed using a multiplate analyzer (Roche Diagnostics). Thrombocyte activity in each condition was measured immediately after sample set-up (Time 0) using the multiplate analyzer and ADP test according to the manufacturer's instructions. Thrombocyte activity was also measured at 3 hour, 6 hour, 9 hour and 22 hour time points. Thrombocyte activity in each condition was normalized to its Time 0 measurement. Data from the six donors were then averaged. The data are shown in Table 2.

TABLE 2
Stabilization of Viable, Activatable Thromobocytes
in a Human Blood Sample for at Least 22 Hours

| Time | | Formulation | Formulation | Formulation | Formulation |
|---|---|---|---|---|---|
| (hr) | Control | A | B | C | D |
| 0 | 100* | 100 | 100 | 100 | 100 |
| 3 | 93 | 110 | 112 | 96 | 98 |
| 6 | 82 | 107 | 111 | 101 | 95 |
| 9 | 77 | 115 | 114 | 101 | 93 |
| 22 | 59 | 91 | 92 | 74 | 90 |

*Values are shown as present activity remaining relative to Time 0

As shown in Table 2, after the 9 hour incubation period, the mean decrease in thrombocyte activity in the NF control condition was −23% compared with +15%, +14%, +1% and −7% for Formulations A, B, C, and D of Table 2, respectively. After a 22 hour incubation period, significant thrombocyte activity was still detected with a mean decrease in thrombocyte activity in the NF control condition of −41%, compared with −9%, −8%, −26% and −10% for Formulations A, B, C, and D of Table 2, respectively.

Formulations of Table 1 comprising a halogenated disaccharide derivative, sucralose, were characterized as described above, and also were identified as possessing stabilizing thrombocyte activity in whole blood for at least 22 hours (Table 3). In this study, these formulations, as set forth in Table 1, were incorporated into hirudin vacuum blood collection tubes prior to blood draw.

TABLE 3
Stabilization of Viable, Activatable Thromobocytes
in a Human Blood Sample for at Least 22 Hours

| Formulation | Time 0 | 3 Hours | 6 Hours | 9 Hours | 22 Hours |
|---|---|---|---|---|---|
| Control | 100 | 89 | 69 | 58 | 60 |
| E | 100 | 100 | 99 | 98 | 97 |
| F | 100 | 94 | 87 | 80 | 73 |

* Values are shown as present activity remaining relative to Time 0

During room temperature blood incubation, thrombocyte activity in the NF control condition decreased by −40% by the 22 hour time point, compared with−3% for Formula E and −27% for Formula F of Table 1.

Unless the context requires otherwise, throughout the present specification and claims, the word “comprise” and variations thereof, such as, “comprises” and “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1.-42. (canceled)

43. A composition for stabilizing thrombocytes at ambient temperatures, comprising:
(i) a di- or tri-chlorinated disaccharide that is present at 0.1-50 μM; and
(ii) an anticoagulant selected from the group consisting of ethylenediaminetetraacetic acid (EDTA), hirudin, heparin, and sodium citrate.

44. The composition of claim 43, wherein the di- or tri-chlorinated disaccharide is sucralose (1,6-dichloro-1,6-dideoxy-β-D-fructofuranosyl-4-chloro-4-deoxy-α-D-galactopyranoside); trichloronated maltose; 1,6-dichloro-1,6-dideoxy-β-D-fructofuranosyl-4-chloro-4-deoxy-6-O-monododecanoate-α-D-galactopyranoside; 1,6-dichloro-1, 6-dideoxy-β-D-fructofuranosyl-4-chloro-4-deoxy-6-O-monotetradecanoate-α-D-galactopyranoside; or any combinations thereof.

45. The composition of claim 43, wherein the di- or tri-chlorinated disaccharide is sucralose (1,6,-dichloro-1,6,dideoxy-β-D-fructofuranosyl-4-chloro-4-deoxy-α-D-galactopyranoside).

46. The composition of claim 43, wherein the anticoagulant is hirudin.

47. The composition of claim 43, wherein the anticoagulant is hirudin, and the di- or tri-chlorinated disaccharide is sucralose (1,6,-dichloro-1,6,dideoxy-β-D-fructofuranosyl-4-chloro-4-deoxy-α-D-galactopyranoside).

48. A composition for stabilizing thrombocytes at ambient temperatures, comprising:
(i) a pH buffer that comprises Tris-HCl, citric acid, or 2×phosphate buffered saline;
(ii) a di- or tri-chlorinated disaccharide; and
(iii) a pentose polyol or a hexose polyol;
wherein the pH of the composition is in the range of about 4.0 to about 9.0.

49. The composition of claim 48, wherein the di- or tri-chlorinated disaccharide is sucralose (1,6-dichloro-1,6-dideoxy-β-D-fructofuranosyl-4-chloro-4-deoxy-α-D-galactopyranoside); trichloronated maltose; 1,6-dichloro-1,6-dideoxy-β-D-fructofuranosyl-4-chloro-4-deoxy-6-O-monododecanoate-α-D-galactopyranoside; 1,6-dichloro-1, 6-dideoxy-β-D-fructofuranosyl-4-chloro-4-deoxy-6-O-monotetradecanoate-α-D-galactopyranoside; or any combinations thereof.

50. The composition of claim 48, wherein the composition further comprises one or both of:
(iv) a functionalized carbohydrate selected from the group consisting of sucralfate and sucrose octasulfate, or a combination thereof; and
(v) a non-reducing sugar selected from the group consisting of sucrose and trehalose, or a combination thereof.

51. A composition for stabilizing thrombocytes at ambient temperatures, comprising:
(i) a pH buffer that comprises Tris-HCl, citric acid, or 2×phosphate buffered saline;
(ii) a di- or tri-chlorinated disaccharide; and
(iii) glycol, glycerol, erythritol, threitol, arabitol, xylitol, ribitol, adonitol, mannitol, sorbitol, galactitol, fucitol, iditol, inositol, or any combinations thereof;
wherein the pH of the composition is in the range of about 4.0 to about 9.0.

52. The composition of claim 51, wherein the di- or tri-chlorinated disaccharide is sucralose (1,6-dichloro-1,6-dideoxy-β-D-fructofuranosyl-4-chloro-4-deoxy-α-D-galactopyranoside); trichloronated maltose; 1,6-dichloro-1,6-dideoxy-β-D-fructofuranosyl-4-chloro-4-deoxy-6-O-monododecanoate-α-D-galactopyranoside; 1,6-dichloro-1, 6-dideoxy-β-D-fructofuranosyl-4-chloro-4-deoxy-6-O-monotetradecanoate-α-D-galactopyranoside; or any combinations thereof.

53. The composition of claim 51, wherein the composition further comprises one or both of:
(iv) a functionalized carbohydrate selected from the group consisting of sucralfate and sucrose octasulfate, or a combination thereof; and
(v) a non-reducing sugar selected from the group consisting of sucrose and trehalose, or a combination thereof.

54. A composition for stabilizing thrombocytes at ambient temperatures, the composition comprising:
(i) a pH buffer that comprises Tris-HCl, citric acid, or 2×phosphate buffered saline;
(ii) ethylenediaminetetraacetic acid (EDTA), hirudin, heparin, sodium citrate, or any combination thereof; and
(iii) a di- or tri-chlorinated disaccharide;
wherein the pH of the composition is in the range of about 4.0 to about 9.0.

55. The composition of claim 54, wherein the composition further comprises one or both of:
(iv) a functionalized carbohydrate selected from the group consisting of sucralfate and sucrose octasulfate, or a combination thereof; and
(v) a non-reducing sugar selected from the group consisting of sucrose and trehalose, or a combination thereof.

56. The composition of claim 54, wherein the composition further comprises a pentose polyol or a hexose polyol.

57. The composition of claim 54, wherein the composition further comprises glycol, glycerol, erythritol, threitol, arabitol, xylitol, ribitol, adonitol, mannitol, sorbitol, galactitol, fucitol, iditol, inositol, or any combinations thereof.

58. The composition of claim 57, wherein the composition further comprises one or both of:
(iv) a functionalized carbohydrate selected from the group consisting of sucralfate and sucrose octasulfate, or a combination thereof; and
(v) a non-reducing sugar selected from the group consisting of sucrose and trehalose, or a combination thereof.

59. The composition of claim 54, wherein the di-or tri-chlorinated disaccharide is sucralose (1,6-dichloro-1,6-dideoxy-β-D-fructofuranosyl-4-chloro-4-deoxy-α-D-galactopyranoside); trichloronated maltose; 1,6-dichloro-1,6-dideoxy-β-D-fructofuranosyl-4-chloro-4-deoxy-6-O-monododecanoate-α-D-galactopyranoside; 1,6-dichloro-1, 6-dideoxy-β-D-fructofuranosyl-4-chloro-4-deoxy-6-Omonotetradecanoate-α-D-galactopyranoside; or any combinations thereof.

60. The composition of claim 59, wherein the composition further comprises a pentose polyol or a hexose polyol.

61. The composition of claim 59, wherein the composition further comprises glycol, glycerol, erythritol, threitol, arabitol, xylitol, ribitol, adonitol, mannitol, sorbitol, galactitol, fucitol, iditol, inositol, or any combinations thereof.

62. The composition of claim 61, wherein the composition further comprises one or both of:
(iv) a functionalized carbohydrate selected from the group consisting of sucralfate and sucrose octasulfate, or a combination thereof; and
(v) a non-reducing sugar selected from the group consisting of sucrose and trehalose, or a combination thereof.
